(19) United States
(12) Patent Application Publication
(10) Pub. No.: US 2026/0256402 A1
(43) Pub. Date: Sep. 3, 2026

(54) EXTERNAL CARDIAC MONITORING SYSTEM

(71) Applicant: Medtronic, Inc., Minneapolis, MN (US)

(72) Inventors: Venkataramana Mohan Pinjala, Hyderabad (IN); Arun Kumar Sathiyamoorthy, Chennai (IN); Shantanu Sarkar, Roseville, MN (US)

(21) Appl. No.: 19/155,857

(22) PCT Filed: Feb. 5, 2024
(86) PCT No.: PCT/US2024/014482

§ 371 Date: Aug. 12, 2025

Related U.S. Application Data
(60) Provisional application No. 63/485,086, filed on Feb. 15, 2023.

(51) Int. Cl. A61B 5/332; A61B 5/00; A61B 5/256; A61B 5/282; A61B 5/308

(52) U.S. Cl. CPC A61B 5/332; A61B 5/256; A61B 5/282; A61B 5/308; A61B 5/6823; A61B 5/6843; A61B 5/721; A61B 2562/0261

(57) ABSTRACT

A medical device system including: a wearable component configured to encircle a portion of a torso of a patient, the wearable component defining a plurality of electrically active regions configured to contact skin of the patient and sense an electrocardiogram (ECG) signal of a heart of the patient and one or more strain gauges. The system further includes a computing module connected to the wearable component and configured to: sense the ECG signal via the plurality of electrically active regions and measure voltage values from the one or more strain gauges over time; determine, based on the voltage values, a respiration signal of the patient and a noise signal in the respiration signal; determine whether the noise signal in the respiration signal satisfies a threshold condition; and based on a determination that the noise signal satisfies the threshold condition, suspend sensing the ECG signal.

This application claims the benefit of U.S. Provisional Patent Application No. 63/485,086, filed Feb. 15, 2023 and entitled “EXTERNAL CARDIAC MONITORING SYSTEM,” the entire contents of which is incorporated herein by reference.

### TECHNICAL FIELD

This disclosure is related to medical devices, and, more particularly, to external medical devices configured to detect signals from a patient.

### BACKGROUND

In some examples, cardiac monitoring devices may be entirely external to the body of a patient. The external cardiac monitoring device may be connected to the skin of the patient, e.g., at a torso of the patient and may sense electrical signals from the heart of the patient without puncturing the skin of the patient. The sensed electrical signals may be used to determine whether the patient is experiencing one or more cardiac conditions and/or other medical conditions.

### SUMMARY

This disclosure describes medical device systems including an external cardiac monitoring (ECM) system configured to be worn by a patient to sense electrical signals (e.g., electrocardiogram (ECG) signals) from a heart of the patient without puncturing skin of the patient. The ECM system may include a wearable component configured to be worn about the body of the patient and a computing device secured to the wearable component and electrically connected to one or more sensors on the wearable component. The computing device of the ECM system may sense the electrical signals from the tissue of the patient via the one or more sensors on the wearable component, e.g., to determine whether the patient is experiencing a cardiac condition (e.g., an arrhythmia, a tachycardia, or the like) based on the sensed electrical signals.

In some examples, this disclosure describes examples methods for determining whether the patient is properly wearing the example medical device system. The computing device may determine noise signals in one or more signals sensed by the medical device system from the patient (e.g., the ECG signals, the respiration signals from the patient) and compare the determined noise signals against threshold conditions. If the computing device determines that the determined noise signals satisfy one or more of the threshold conditions, the computing device may suspend, temporarily cease, sensing the electrical signals from the heart of the patient.

The example devices, systems, or methods described in this disclosure provides several benefits over other external cardiac monitoring devices and/or systems. In some examples, the wearable component may allow the medical device system to continuously monitor and/or sense electrical signals from the patient without a loss of connection between the patient and the sensors of the wearable component. In some examples, the medical device system may allow for re-use the wearable component and/or the computing device of the medical device system with the patient and/or with multiple patients. In some examples, the example method of determining whether the medical device system is detecting a threshold amount of noise signals from the patient (e.g., in the sensed electrical signals and/or one or more other sensed signals) and suspending the sensing of the electrical signals if the medical device system detects the threshold amount of noise signals may prevent the medical device system from storing erroneous and/or inaccurate electrical signals and may improve the accuracy of the determination of whether the patient is experiencing a cardiac condition.

In some examples, this disclosure describes a medical device system comprising: a wearable component configured to encircle a portion of a torso of a patient, the wearable component defining a longitudinal axis and comprising: an electrically conductive fabric; a plurality of electrically active regions defined by the electrically conductive fabric and disposed along the longitudinal axis of the electrically conductive fabric, each of the plurality of electrically active regions being configured to contact skin of the patient and sense an electrocardiogram (ECG) signal of a heart of the patient; one or more strain gauges disposed within the electrically conductive fabric and along the longitudinal axis; and a recess; and a computing module disposed within the recess, the computing module comprising: sensing circuitry electrically connected to the plurality of electrically active regions and the one or more strain gauges; and processing circuitry configured to: cause the sensing circuitry to sense the ECG signal via the plurality of electrically active regions and measure voltage values from the one or more strain gauges over time; determine, based on the voltage values, a respiration signal of the patient and a noise signal in the respiration signal; determine whether the noise signal in the respiration signal satisfies a threshold condition; and based on a determination that the noise signal satisfies the threshold condition, cause the sensing circuitry to suspend sensing the ECG signal.

In some examples, this disclosure describes a medical device system comprising: a wearable component configured to encircle a portion of a torso of a patient, the wearable component defining a longitudinal axis and comprising: an electrically conductive fabric; a plurality of electrically action regions defined by the electrically conductive fabric and disposed along the longitudinal axis of the electrically conductive fabric, each of the plurality of electrically active regions being configured to contact skin of the patient and sense an electrocardiogram (ECG) signal of a heart of the patient; and a recess; and a computing module disposed within the recess, the computing module comprising: sensing circuitry electrically connected to the plurality of electrically active regions; and processing circuitry configured to: cause the sensing circuitry to sense the ECG signal via the plurality of electrically active regions; determine a noise signal within the sensed ECG signal; determine whether the noise signal satisfies a threshold condition; and based on a determination that the noise signal satisfies the threshold condition, cause the sensing circuitry to suspend sensing the ECG signal.

In some examples, this disclosure describes a computing device configured to sense an electrocardiogram (ECG) signal from a patient, the computing device comprising: sensing circuitry comprising one or more sense amplifiers; processing circuitry configured to: sense, via the sensing circuitry, a signal from one or more sensors on a wearable component in contact with skin of the patient; determine a noise signal within the sensed signal; determine whether the noise signal satisfies a threshold condition; determine, based on a determination that the noise signal satisfies the threshold condition, that the patient is improperly wearing the wearable strap; and based on a determination that the patient is improperly wearing the wearable component, cause the sensing circuitry to suspend sensing the ECG signal; and a fixation mechanism configured to electrically connect the computing device the one or more sensors.

In some examples, this disclosure describes a method comprising: sensing, by sensing circuitry of a computing module and via a plurality of electrically active regions disposed on a wearable component worn by a patient and configured to contact skin of the patient, an electrocardiogram (ECG) signal of a heart of the patient, wherein the wearable component is configured to encircle a portion of a torso of the patient, and wherein the wearable component comprises: an electrically conductive fabric defining the plurality of electrically active regions along a longitudinal axis of the wearable component; and a recess configured to retain the computing module; determining, by processing circuitry of the computing module and based on the ECG signal, a noise signal within the ECG signal; determining, by the processing circuitry, whether the noise signal satisfies a threshold condition; determining, by the processing circuitry and based on a determination that the noise signal satisfies the threshold condition, that the patient is improperly wearing the wearable component; and based on a determination that the patient is improperly wearing the wearable component, causing, by the processing circuitry, the sensing circuitry to suspend sensing the ECG signal.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example medical device system including an external cardiac monitoring system.

FIG. 2A is a conceptual diagram illustrating a front view of the external cardiac monitoring system of FIG. 1.

FIG. 2B is a conceptual diagram illustrating a rear view of the external cardiac monitoring system of FIG. 1.

FIG. 2C is a conceptual diagram illustrating a top view of the external cardiac monitoring system of FIG. 1.

FIG. 2D is a conceptual diagram illustrating a front view of the external cardiac monitoring system of FIG. 1 without a computing device of the external cardiac monitoring system.

FIG. 3A is a conceptual diagram illustrating an outer surface of a front housing of the computing device of FIG. 1.

FIG. 3B is a conceptual diagram illustrating an inner surface of the front housing of the computing device of FIG. 1.

FIG. 3C is a conceptual diagram illustrating an outer surface of a rear housing of the computing device of FIG. 1.

FIG. 3D is a conceptual diagram illustrating an inner surface of the rear housing of the computing device of FIG. 1.

FIG. 4A is a conceptual diagram illustrating a front view of the external cardiac monitoring system of FIG. 1 with a plurality of expandable members.

FIG. 4B is a conceptual diagram illustrating a top view of the external cardiac monitoring system of FIG. 4A.

FIG. 5 is a functional block diagram illustrating an example configuration of the computing device of FIG. 1.

FIG. 6 is a conceptual diagram illustrating an example medical device system including the example cardiac monitoring system of FIG. 1 and one or more external monitoring devices.

FIG. 7A is a flow diagram illustrating an example method of sensing signals from a patient using an example medical device system.

FIG. 7B is a flow diagram illustrating an example method of expanding expandable members of an example medical device system.

FIG. 8 is a flow diagram illustrating another example method of sensing electrical signals from a patient using an example medical device system.

### DETAILED DESCRIPTION

This disclosure describes external cardiac monitoring (ECM) systems, components of ECM systems, medical device systems including ECM systems, and related techniques of using and forming ECM systems. An example ECM system may include a wearable component (e.g., a wearable strap, a wearable vest, or the like) including one or more sensors (e.g., an electrode, an electrically active region defining an electrode) placed in contact with the skin of the patient and a computing device secured to the wearable component. The computing device may sense electrical signals of the heart of the patient through the skin of the patient via the one or more sensors and may store, transmit, and/or analyze the sensed electrical signals (e.g., to determine whether the patient is experiencing a cardiac condition). The computing device may determine, based on the sensed electrical signals and/or one or more other sensed signals from the patient (e.g., a respiration signal of the patient), whether the patient is properly using the ECM system and may suspend sensing of the electrical signals to prevent recordation and/or use of inaccurate sensed electrical signals.

A medical device system may include an example ECM system and use the ECM system to monitor the heart of the patient and sense electrical signals (e.g., in the form of electrocardiogram (ECG) signals) from the heart of the patient. The use of an external monitoring system, such as the ECM systems described in this disclosure, may allow the patient to attach and/or remove components of the ECM system (e.g., the wearable component, one or more sensors) from the body of the patient. The use of the external monitoring systems may also reduce a financial cost of the medical device system and reduce and/or eliminate a need to implant a medical device within the body of the patient. Although the sensed electrical signals is primarily described with reference to ECG signals, other electrical signals corresponding to the cardiac activity of the heart of the patient may also be sensed by the example ECM systems and used to make one or more of the example determinations made by the ECM systems described herein. For example, impedance, sounds from the heart, accelerations detected by a multi-axis accelerometer (e.g., a three-axis accelerometer), respiration activity (e.g., detected by strain gauge(s), and/or pulse oximetry (e.g., detected by optical system(s)) may be used to make any of the example determinations made by the ECM systems described herein.

Other external monitoring systems may use adhesives (e.g., adhesive patches) to secure the one or more sensors of the external monitoring system to the body of the patient. The use of adhesives may limit an amount of time a patient may wear the external monitoring system, cause patient discomfort, and/or limit reusability of the components of the external monitoring system. For example, the adhesives and the one or more sensors of the external monitoring system may not be reusable and may need to be replaced during subsequent use of the external monitoring system.

In some examples, the one or more sensors of the external monitoring system may be caused to move around the body of the patient and/or lose contact with the skin of the patient. The movement or loss of contact of the one or more sensors may cause the external monitoring system to fail to sense the ECG signals of the heart or to sense inaccurate ECG signals (e.g., ECG signals not representative of the condition of the heart, ECG signals altered and/or distorted by the position of the one or more sensors relative to the heart).

The example ECM systems described in this disclosure include a wearable component including or defining one or more sensors. The one or more sensors may include but are not limited to, an electrode, an electrically active region defining an electrode, strain gauge(s), capacitors, or the like. The wearable component may be worn, hooked, wrapped, or otherwise removably secured around the body of the patient (e.g., around the torso of the patient). When worn by the patient, the wearable component may place one or more of the sensors (e.g., an electrode) in contact with and/or adjacent to the skin of the patient at one or more predetermined locations.

The example ECM systems may include a computing device removably secured to the wearable component, e.g., within a recess defined by the wearable component. When secured to the wearable component, the computing device may be electrically connected to the one or more sensors of the wearable component and sense one or more signals (e.g., the ECG signal, a respiration signal) from the patient. The wearable component and the computing device may be re-used by the patient or by another patient without requiring the use of any new components.

In some examples, the computing device determines whether the patient is properly wearing the ECM system and/or whether the one or more sensors are placed in contact with the body of the patient at predetermined locations. The computing device may determine a noise signal in the sensed signal(s) and determine that the one or more sensors are not placed at the predetermined locations based on a determination that the noise signal satisfies one or more threshold noise conditions.

If the computing device of the ECM system determines that the one or more sensors are not placed at the predetermined locations and/or that the wearable component is improperly worn, the computing device may suspend sensing of the ECG signals from the patient, e.g., to prevent storage, transmission, and/or use of inaccurate, altered, and/or distorted ECG signals. The computing device may suspend the sensing of the ECG signals for a predetermined period of time, until the noise signal no longer satisfies the one or more threshold conditions, in response to an user input, or like. In some examples, the computing device may output a notification and/or alert to the user to communicate to the user a need to adjust the wearable component. The computing device may be configured to be able to resume the sensing of the ECG signals, e.g., to allow the patient to re-adjust the ECM system and/or the one or more sensors about the body of the patient. In some examples, the computing device suspends the sensing of the electrical signals by saturating one or more sensing components (e.g., sense amplifiers) of the computing device, temporarily powering off the computing device, transmitting a notification to an external device accessible by the patient or a clinician, or the like.

Suspension of the sensing of the ECG signals in response to the determination that the one or more sensors are not placed at the predetermined locations on the body of the patient may provide several advantages over other external monitoring systems. The suspension of the sensing of the ECG signals may increase battery life or power efficiency of the computing device by reducing and/or preventing the sensing, transmission, and/or analysis of inaccurate or distorted ECG signals. The suspension may also improve ECG storage capacity of the computing device by reducing and/or preventing storage of inaccurate or distorted ECG signals in memory of the computing device. The suspension may also increase the accuracy of any determinations (e.g., of the patient experiencing a cardiac condition) by the computing device and/or one or more other computing devices and/or systems of an example medical device system by reducing and/or preventing the use of lower-quality, inaccurate, and/or distorted ECG signals in the determinations.

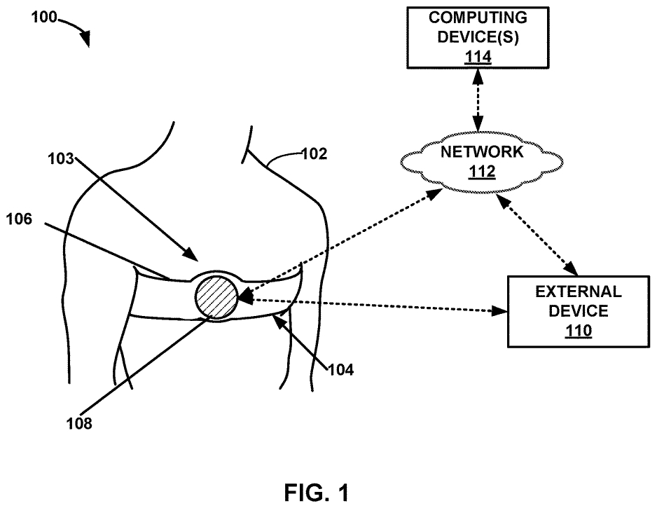

FIG. 1 is a conceptual diagram illustrating an example medical device system 100 including an external cardiac monitoring (ECM) system 104. As illustrated in FIG. 1, medical device system 100 includes an ECM system 104 including a wearable component 106 and a computing device 108 secured to wearable component 106. Computing device 108 may communicate with an external device 110, a network 112, and/or one or more computing device(s) 114 via network 112.

Wearable component 106 is configured to be worn by patient 102, e.g., around torso 103 of patient 102. Wearable component 106 may include, but is not limited to, a wearable strap, a wearable vest, a wearable belt, wearable suspenders, or any other wearable design configured to place sensors in contact with the skin of patient 102 and around torso 103. Wearable component 106 includes one or more sensors configured to sense signals from patient 103 corresponding to physiological metrics of patient 103. Each of the one or more sensors may include one or more sensing components (e.g., electrodes). For example, the one or more sensors may sense electrical signals (e.g., ECG signals) corresponding to electrical activity of the heart of patient 102, respiration signals corresponding to a respiration rate (RR) of patient 102, or the like. The one or more sensors may include, but are not limited to, electrodes, electrically-active regions defining electrodes, strain-gauge sensors, capacitors, or one or more other sensors configured to sense electrical activity of the heart of patient 102 and/or respiratory activity of patient 102. While the sensors described herein are primarily placed around torso 103, in some examples the one or more sensors may be placed at other locations on the body of patient 102 (e.g., around the shoulder of patient 102).

Wearable component 106 places at least some of the one or more sensors (e.g., one or more electrodes) in contact with the skin of patient 102, e.g., at predetermined positions on torso 103 of patient 102. Wearable component 106 places the sensors in contact with the skin without the use of adhesives or any other fixation mechanism. Wearable component 106 may be worn or removed by patient 102 and may be re-used by patient 102 or by one or more other individuals (e.g., another patient).

Computing device 108 includes computing circuitry (e.g., sensing circuitry, processing circuitry) disposed within a housing removable secured to wearable component 106, e.g., within a recess defined by wearable component 106. Computing device 108 may be electrically connected to the one or more sensors on wearable component 106 and may be configured to sense signals from patient 102 via the one or more sensors on wearable component 106. Computing device 108 may sense ECG signals from the heart of patient 102 via the one or more sensors on wearable component 106 and in memory of computing device 108 and/or transmit the sensed ECG signals to external device 110, network 112, and/or computing device(s) 114. In some examples, computing device 108 determines, based on the sensed ECG signals, whether patient 102 has experienced, is experiencing, and/or will experience a cardiac condition (e.g., arrhythmia, tachycardia, or the like). In some examples, computing device 108 may determine, based on the sensed signals, other cardiac conditions including, but are not limited to, decompensated heart failure, exacerbations of chronic obstructive pulmonary disease (COPD), or the like. Based on the determination that the patient 102 has experienced, is experiencing, and/or will experience the cardiac condition, computing device 108 may transmit a notification including the determinations and/or the sensed electrical signals to external device 110, network 112, and/or computing device(s) 114.

Computing device 108 determines, based on sensed signals (e.g., sensed ECG signals, sensed respiration signals) from patient 102, whether wearable component 106 is properly worn by patient 102 and/or whether the one or more sensors of wearable component 106 are placed on torso 103 at predetermined locations. For example, computing device 108 may determine whether electrodes on wearable component 106 are placed on torso 103 at the predetermined locations based on the sensed signals. In some examples, computing device 108 determines noise signals in the sensed signals (e.g., a noise signal in the ECG signal, a noise signal in the sensed respiration signal). Based on whether the determined noise signals satisfy one or more threshold noise conditions, computing device 108 determines whether wearable component 106 is properly worn by patient 102 and/or whether the one or more sensors are misplaced on patient 102.

When the wearable component 106 is improperly worn, one or more sensors of wearable component 106 may be misplaced on patient 102, computing device 108 may sense the ECG signals from patient 102 that are inaccurate, altered, or otherwise distorted (e.g., not representative of the signals of the heart of patient 102). Based on the determination by computing device 108 that wearable component 106 is improperly worn by patient 102, computing device 108 may suspend the sensing of ECG signals from patient 102. Computing device 108 may suspend the sensing of ECG signals from patient 102 to preserve power and/or storage space and to prevent inaccurate determinations, by computing device 108, external device 110, network 112, and/or computing device(s) 114 of condition of patient 102 based on the sensed ECG signals. In some examples, computing device 108 may output a signal (e.g., an auditory, tactile, or visual signal) to alert patient 102 that computing device 108 is suspending the sensing of ECG signals and/or that wearable component 106 needs to be adjusted.

Computing device 108 may temporarily suspend the sensing of the ECG signals. Computing device 108 may suspend the sensing of the ECG signals for a predetermined amount of time, until reception of user input to resume via external device 110, network 112, and/or computing device(s) 114, until computing device 108 determines that the noise signals of the sensed signals no longer satisfy any of the threshold noise conditions, or until reception of other information and/or inputs by system 100. Computing device 108 may suspend the sensing of the ECG signals by temporarily powering off computing device 108, or suspending sensing functions of sensing circuitry of computing device 108 (e.g., by saturating one or more sense amplifiers of the sensing circuitry of computing device 108). In some examples, computing device 108 may turn off the automatic detection of cardiac conditions by the processing circuitry. Computing device 108 may perform any other actions to cause the sensing circuitry of computing device 108 to suspend sensing of the ECG signals, to cause the processing circuitry of computing device 108 to suspend storing of any sensed ECG signals in the memory of computing device 108, and/or to cause the processing circuitry of computing device 108 to suspend transmitting any sensed ECG signals to external device 110, network 112, and/or computing device(s) 114.

In some examples, wearable component 106 includes one or more expandable members (not pictured) disposed along wearable component 106. The one or more expandable members may be configured to be in fluid communication with a blower disposed on computing device 108. In response to the determination that wearable component 108 is improperly worn by patient 102, computing device 108 may engage the blower to expand the one or more expandable members from a collapsed configuration to an expanded configuration. In the expanded configuration, the one or more expandable members increase a force applied on the one or more sensors to increase contact between the one or more sensors and the skin of patient 102, e.g., to improve sensing of ECG signals from patient 102. Computing device 108 may expand the one or more expandable members prior to, instead of, or in conjunction with suspending sensing of the ECG signals.

External device 110 may be a computing device accessible by patient 102 and configured to communicate with computing device 108. Computing device(s) 114 may include one or more computing devices configured to communicate with computing device 108 and/or external device 110 via network 112. Computing device(s) 114 may not be directly accessible by patient 102. Patient 102 may operate computing device 108 via external device 110. One or more other individuals (e.g., a clinician) may operate computing device 108 via one or more computing device(s) 114. External device 110 and/or computing device(s) 114 may include, but are not limited to, a personal computer, a laptop computer, a tablet, a smartwatch, a smartphone, or one or more other computing devices.

Network 112 may include one or more cloud computing networks or cloud computing environments in communication with computing device 108 of ECM system 104, external device 110 and/or one or more computing device(s) 114. External device 110 and/or computing device(s) 114 may communicate with computing device 108 directly or via network 112.

External device 110, network 112, and/or computing device(s) 114 may receive information from computing device 108 (e.g., information corresponding to the sensed electrical signals, information corresponding to determinations made by computing device 108) to patient 102 and/or one or more other individuals (e.g., a clinician). External device 110, network 112, and/or computing device(s) 114 may determine, based on the received information, whether wearable component 106 is worn properly by patient 102 and/or whether patient 102 is experiencing, has experienced, or will experience a cardiac condition.

External device 110 and/or computing device(s) 114 may receive user inputs from patient 102 and/or the one or more other individuals and transmit the received user inputs to computing device 108. The user inputs may include, but are not limited to, instructions to computing device 108 to suspend sensing of the ECG signals, instructions to computing device 108 to resume the sensing of the ECG signals, or a request to show real-time ECG signals from computing device 108.

Network 112 and/or computing device(s) 114 may receive information from external device 110 and/or computing device 108 (e.g., information corresponding to the sensed ECG signals, the sensed ECG signals, information corresponding to the determination made by computing device 108, that patient 102 has experienced, is experiencing, or will experience a cardiac condition.

For the purposes of this disclosure, a “front view”, “front side”, or “front housing” of any components of medical device system 100 (e.g., of wearable component 106, of computing device 108) refers to a view or side of the component that, when worn by patient 102, faces away from patient 102. For the purposes of this disclosure, a “rear view,” “rear side,” or “rear housing of any components of medical device system 100 (e.g., of wearable component 106, of computing device 108) refers to a view or side of the component that, when worn by patient 102, faces towards patient 102.

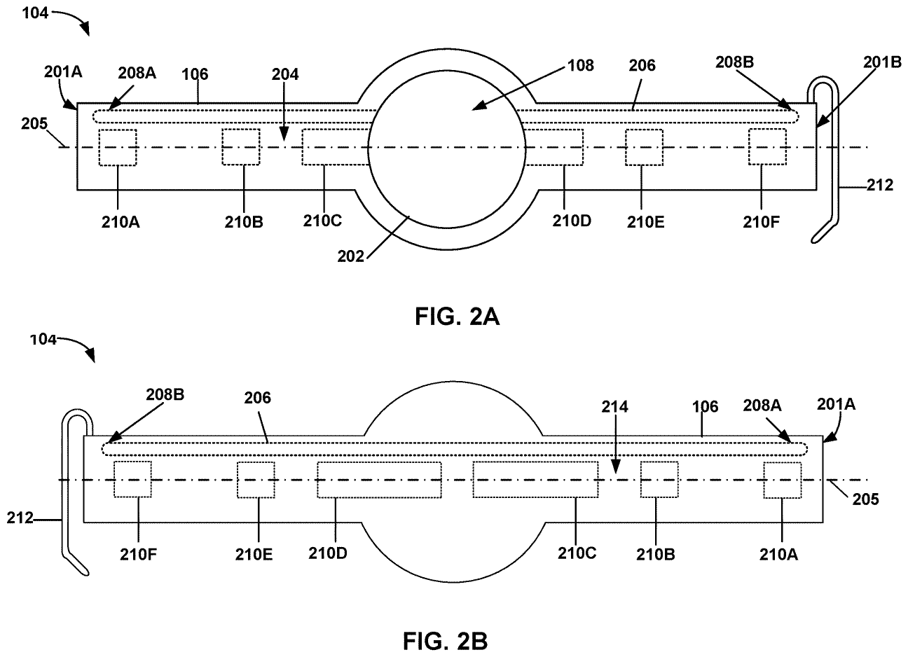

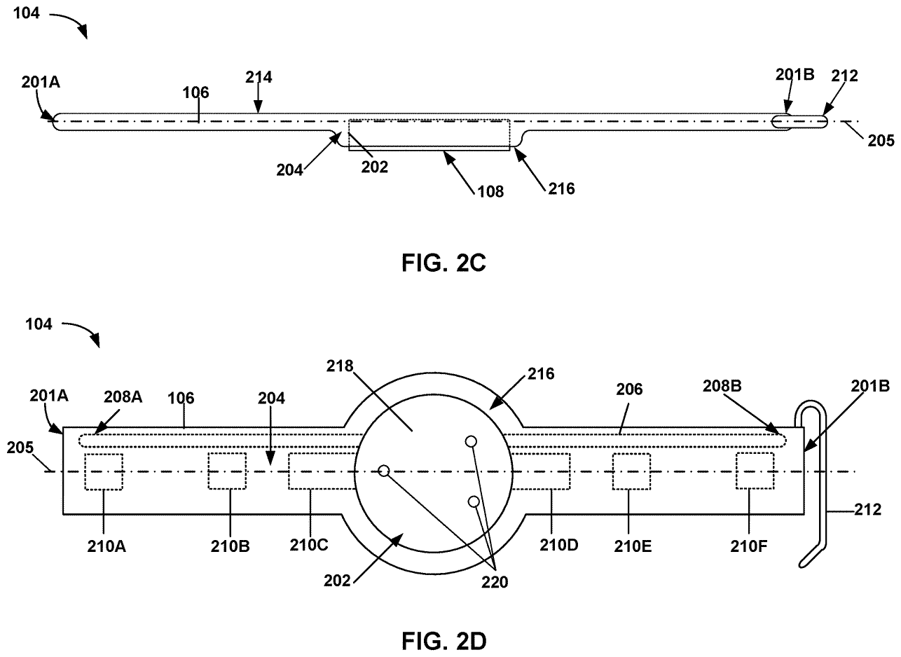

FIG. 2A is a conceptual diagram illustrating a front view of ECM system 104 of FIG. 1. ECM system 104 includes wearable component 106 extending from a first end 201A to a second end 201B. Wearable component 106 includes one or more sensors disposed on or defined by wearable component 106 and a fixation component 212 dispose on an end of ECM system 104 (e.g., on first end 201A or second end 201B). Wearable component 106 may define a recess 202 configured to receive computing device 108.

Wearable component 106 includes a flexible material 204 (e.g., a fabric) extending from first end 201A to second end 201B. Flexible material 204 include one or more sensors disposed within flexible material 204, within a recess formed by flexible material 204, and/or on an outer surface of flexible material 204. For example, wearable component 106 includes strain gauge(s) 206 dispose within flexible material 204 and along longitudinal axis 205 extending from first end 201A towards second end 201B. In some examples, wearable component 106 includes one or more electrodes 210A-210F (collectively referred to as “electrodes 210”) disposed along longitudinal axis 205. In some examples, wearable component 106 may include a Zephyr™ strap of a Zephyr™ Performance System available from Medtronic Plc., Dublin, Ireland.

Flexible material 204 may include a dry-electrode material (e.g., a dry conductive cloth) or another electrically conductive material. The dry-electrode material may include one or more electrically active regions, each electrical active regions defining one of electrodes 210. Flexible material 204 may be configured to elastically extend, compress, and/or or twist, e.g., in response to movement of patient 102. Flexible material 204 may include a central portion defining recess 202. Recess 202 may receive computing device 108 and include components configured to secure computing device 108 to wearable component 106 and to electrically connect computing device 108 to the one or more sensors in wearable components 106, e.g., to strain gauge(s) 206 and/or electrodes 210. When worn by patient 102, wearable component 106 may place computing device 108 across sternum of patient 102 (e.g., as illustrated in FIG. 1), along the side of torso 103 of patient 102, over the back of patient 102, and/or any other location on the body of patient 102. Flexible material 204 may be at least partially porous, e.g., to increase patient comfort.

In some examples, as illustrated in FIG. 2A, flexible material 204 may elastically deform to fit different body shapes of torso 103. In some examples, wearable component 106 may include straps, zippers, and/or buckles configured to adjust the dimensions of wearable component 106 and/or flexible material 204 to conform to torso 103 of patient 102.

Electrodes 210 may be positioned along longitudinal axis 205 such that when wearable component 106 is worn by patient 102, electrodes 210 are in contact with the skin of patient 102 at predetermined locations around torso 103 of patient 102. Each of electrodes 210 may be configured to sense ECG signals from the skin of patient 102 and to transmit the sensed ECG signals to computing device 108 via one or more conductors and/or conductive channels in flexible material 204. In some examples, wearable component 106 includes two or more conductors disposed within wearable component 106. In some examples, two of electrodes 210 (e.g., electrode 210A, electrode 210F) may be configured to be capacitors and may generate an electric field between the two electrodes 210. A capacitance of the generated electric field may vary as a result of the expansion and contraction of torso 103 in response to respiration of patient 102. Computing device 108 may sense the capacitance of the electric field and changes in the capacitance via the two electrodes 210.

Strain gauge(s) 206 may extend along longitudinal axis 205 of flexible material 204 from first end 208A to second end 208B. When patient 102 respirates while wearing ECM wearable component 106, torso 103 of patient 102 may expand or contract, which applies strain or stress to strain gauge(s) 206 along longitudinal axis 205. The applied strain or stress causes strain gauge(s) 206 to output a voltage value which may be detected by computing device 108.

Fixation component 212 is configured to secure one end of wearable component 106 (e.g., second end 201B) to the other end of wearable component 106 (e.g., first end 201A). In some examples, each end of wearable component 106 may include a fixation component 212 configured to be removably secured to another fixation component 212 on the other end of wearable component 106. Fixation component 212 may include but is not limited to, Velcro straps, latches, buttons, zippers, pins, straps, buckles, or any other fixation device configured to secure multiple pieces of fabric.

FIG. 2B is a conceptual diagram illustrating a rear view of ECM system 104 of FIG. 1. As illustrated in FIG. 2B, a rear surface 214 of wearable component 106 does not include any protrusions and/or indentations in some examples, and is configured to contact the skin of patient 102 across at least a portion of rear surface 214. FIG. 2C is a conceptual diagram illustrating a top view of the ECM system 104 of FIG. 1. Wearable component 106 may include protrusion 216 extending away from the front surface of wearable component 106 and defining recess 202. Recess 202 may extend into protrusion 216 and towards rear surface 214. Recess may be a blind opening configured to receive computing device 108. When computing device 108 is disposed within recess 202, computing device 108 may partially protrude from recess 202, e.g., to facilitate insertion and/or removal of computing device 108 from recess 202. In some examples, a front surface of computing device 108 may be flush with a front surface of protrusions 216.

FIG. 2D is a conceptual diagram illustrating a front view of the ECM system 104 of FIG. 1 without computing device 108. As illustrated in FIG. 2D, recess 202 extends from the front surface of protrusion 216 to an inner surface 218 of recess 202. A plurality of electrical contacts 220 are disposed on the inner surface 218 and configured to engage with one or more fixation mechanisms on computing device 108 to electrically connect electrodes 210, strain gauge(s) 206, and/or one or more other sensors in wearable component 106. In some examples, each of electrical contacts 220 may correspond to two or more sensors in wearable component 106. In some examples, each of electrical contacts 220 may correspond to a single sensor (e.g., to a single electrode of electrodes 210, to a single strain gauge 206), or to a single type of sensor (e.g., electrodes 210 only, strain gauges 206 only) in wearable component 106.

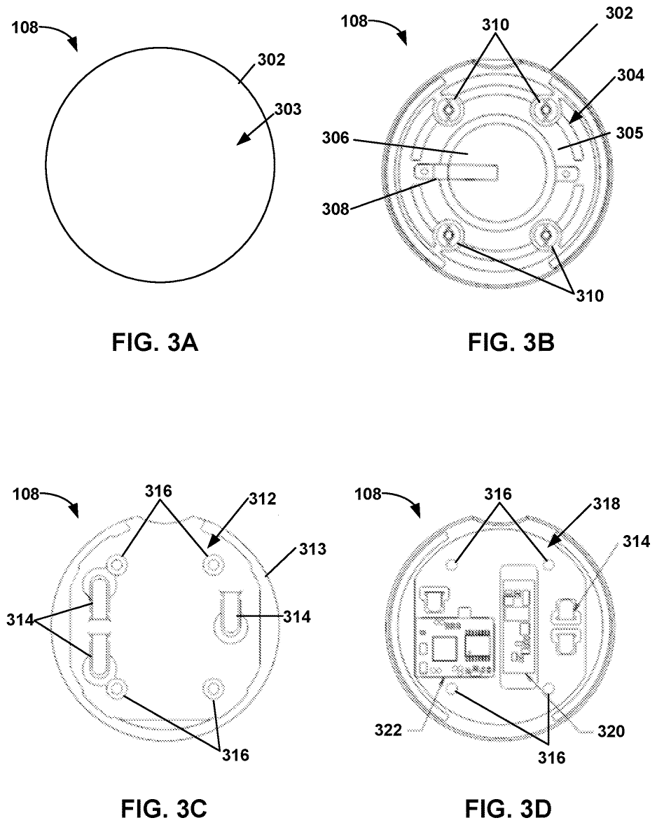

FIG. 3A is a conceptual diagram illustrating an outer surface 303 of a front housing 302 of computing device 108 of FIG. 1. FIG. 3B is a conceptual diagram illustrating an inner surface 304 of front housing 302 of computing device 108 of FIG. 1. FIG. 3C is a conceptual diagram illustrating an outer surface 312 of a rear housing 313 of computing device 108 of FIG. 1. FIG. 3D is a conceptual diagram illustrating an inner surface 318 of rear housing 313 of computing device 108 of FIG. 1. As illustrated in FIGS. 3A-3D, front housing 302 and rear housing 313 may be removably affixed to one another to define a housing containing computing circuitry and/or other components of computing device 108 as described in this disclosure. Front housing 302 and rear housing 313 may be separated and/or united, e.g., to allow access to the computing circuitry and/or a power source within computing device 108. In some examples, a user (e.g., patient 102, a clinician) may separate front housing 302 and rear housing 313 to replace a removable power source disposed within the housing of computing device 108.

In some examples, the outer surface of front housing 302 may include one or more of indentations, protrusions, buttons, knobs, or any other interfaces and/or control, e.g., to allow patient 102 to interact with and/or transmit user inputs to computing device 108 or to facilitate separation of front housing 302 and rear housing 313.

Inner surface 304 of front housing 302 may include power source 306 of computing device 108 and one or more inserts 310 extending from inner surface 304 and towards rear housing 314. Power source 306 may include a removable power source such as, but is not limited to, a coin cell battery, a button battery, or the like. Power source 306 may be removably secured to inner surface 304 by clip 308. Clip 308 may be elastically deformed to allow for removal of power source 306 from between clip 308 and inner surface 304. In some examples, as illustrated in FIG. 3B, clip 308 may be electrically conductive and inner surface 304 may include electrically conductive contacts 305 to electrically connect power source 306 to inserts 310 and/or computing circuitry and/or other components within computing device 108.

Insert(s) 310 may include electrically conductive materials configured to electrically connect power source 306 and/or contacts 305 to computing circuitry and/or other components within computing device 108 and/or to the inner surface 318 of rear housing 313. In some examples, insert(s) 310 may extend to contact and electrically connect with wearable component 106 (e.g., with electrical contacts 220 with recess 202). Front housing 302 may include one or more insert(s) 310.

In some examples, power source 306 may be permanently disposed within computing device 108 and may be recharged via an external port, via wireless charging (e.g., inductive charging, or the like. In some examples, power source 306 may be disposed within a separate compartment and may be removed, e.g., without separating front housing 302 and rear housing 313.

Outer surface 312 of rear housing 313 may be configured to be in contact with inner surface 218 of wearable component 106 when computing device 108 is disposed within recess 202. Rear housing 313 includes fixation mechanism(s) 314 extending away from outer surface 312 Fixation mechanism(s) 314 may engage with inner surface 218 of recess 202 to removably secure computing device 108 to wearable component 106. In some examples, fixation mechanism(s) 314 are electrically connected to power source 306 and computing circuitry and/or other components of computing device 108 and may transmit electrical signals and/or receive electrical signals from wearable component 106. In some examples, fixation mechanism(s) 314 are electrically connected to (e.g., in contact with) electrical contacts 220.

In some examples, as illustrated in FIG. 3C, fixation mechanism(s) 314 may include spring clips. In some examples, fixation mechanism(s) 314 may include locking lug(s), locking screw(s), locking recess(s), and/or one or more other fixation mechanisms and/or devices configured to removably secure computing device 108 within recess 202.

Rear housing 313 may include one or more contact(s) 316 configured to electrically connect computing device 108 to wearable component 106 (e.g., to sensors of wearable component 106). Contact(s) 316 may include electrically-conductive material configured to transmit electrical signals between computing device 108 and wearable component 106. In some examples, contact(s) 316 may include openings in rear housing 313 configured to allow portions of insert(s) 310 to extend through rear housing 313 to electrically connect to contact(s) 316.

Contact(s) 316, fixation mechanism(s) 314, and computing circuitry of computing device 108 may be disposed on inner surface 318 of rear housing 313. Inner surface 318 may include electrical contacts configured to electrically connect contact(s) 316, fixation mechanism(s) 314, and the computing circuitry of computing device 108. In some examples, as illustrated in FIG. 3D, the computing circuitry of computing device 108 may be separated into separate computing modules (e.g., sensing module 320 containing sensors and sensing circuitry, processing module 322 containing processing circuitry). In some examples, computing circuitry of computing device 108 may be disposed in a single computing module. In some examples, each of electrodes 210 may be electrically connected to a separate sensing module 320. In some examples, electrodes 210 may be electrically connected to a single sensing module 320.

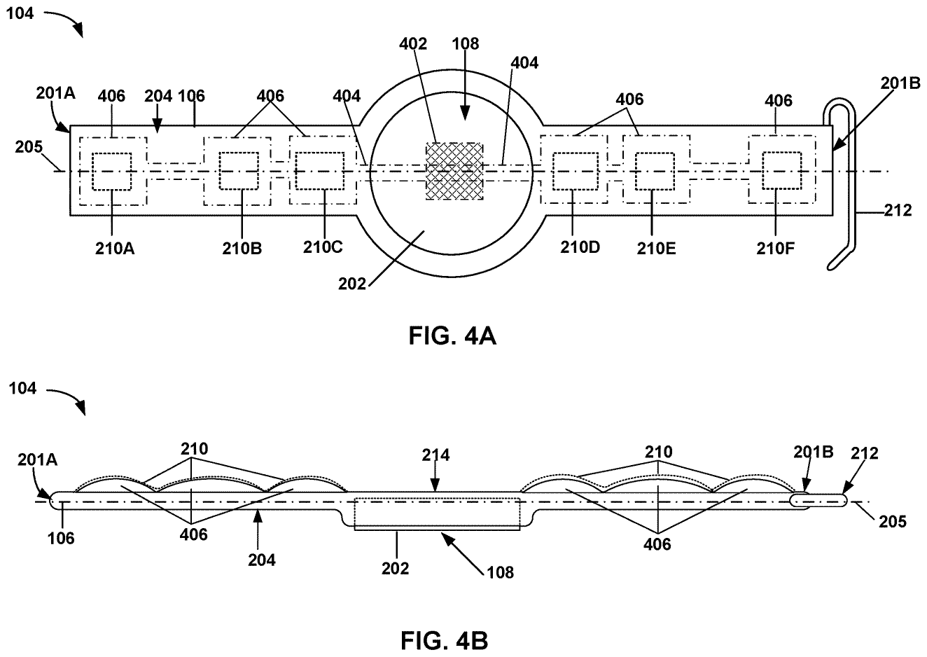

FIG. 4A is a conceptual diagram illustrating a front view of ECM system 104 of FIG. 1 with a plurality of expandable members 406. Expandable members 406 may be configured to transform between a collapsed configuration and an expanded configuration. FIG. 4B is a conceptual diagram illustrating a top view of ECM system 104 of FIG. 4A. In such examples, each of electrodes 210 may be disposed on rear surface 214 and over one or more of expandable members 406. As illustrated in FIG. 4B, when expanded, expandable members 406 may urge electrodes 210 towards the skin of patient 102, e.g., to increase a contact force between electrodes 210 and the skin of patient 102. The increased contact force may increase sensing capabilities and/or sensing sensitivity of electrodes 210, e.g., by improving contact between electrodes 210 and the skin.

As illustrated in FIG. 4A, each of expandable members 406 is in fluid communication with recess 202 and/or computing device 108 via channels 404. Computing device 108 may include a blower 402 (alternatively referred to as “fan 402,” “compressor 402,” or “pump 402”). When computing device 108 is secured within recess 202, one or more output channels of blower 402 may align with and/or fluidically connect with channels 404.

In some examples, blower 402 may be disposed on wearable component 106 (e.g., within recess 202). In such examples, blower 402 may be in fluid communication with channels 404 and insertion of computing device 108 may electrically connect blower 402 and computing device 108 (e.g., via contacts 220). Once blower 402 and computing device 108 are electrically connected, computing device 108 may transmit instructions (e.g., via electrical signals) to blower 402 to cause blower 402 to output air to expandable members 406 (e.g., to expand expandable members 406 to the expanded configuration, to power off, and/or to suck air from expandable members 406 (e.g., to collapse expandable members 406 to the collapsed configuration).

In some examples, blower 402 may take in air from outside of ECM system 104 (e.g., via one or more openings in computing device 108 and/or wearable component 106). Blower 402 may then output the air into expandable members 406 via channels 404 to cause expandable members 406 to expand into expanded configurations. Blower 402 may continue to output air into expandable members 406 until computing device 108 determines that expandable members 406 are in the expanded configurations. Computing device 108 may determine that expandable members 406 are in the expanded configurations based on information and/or signals from one or more sensors on wearable component 106 (e.g., from electrodes 210), based on a determination that blower 402 has outputted air into expandable members 406 for a threshold period of time, based on a determination that blower 402 has outputted a threshold volume of air into expandable members 406, or the like.

Each of expandable members 406 may be defined by flexible material 204 and may include an inner volume that may be caused to expand by introduction of air through channels 404. In some examples, each of expandable members 406 may disposed within a region in wearable component 106 and between two or more layers of flexible materials 204. In some examples, expandable members 406 may be at least partially permeable or semi-permeable and blower 402 may continue to output air to expandable members 406 to maintain expandable members 406 in the expanded configurations. After blower 402 is powered off, expandable members 406 may automatically transform back to the collapsed configuration via diffusion of air out of the inner volumes of expandable members 406. In some examples, blower 402 may suck air from the inner volume of expandable members 406 via channels 404 to transform expandable members 406 from the expanded configurations to the collapsed configurations.

Each of expandable members 406 may be disposed under or otherwise adjacent to one or more of electrodes 210. In some examples, as illustrated in FIGS. 4A and 4B, electrically active portions of flexible material 204 (e.g., electrically action portions of flexible material 204 defining electrodes 210) may at least partially define the inner volumes of expandable member 406. In other examples, electrodes 210 may dispose on rear surface 214 of wearable component 106 and over expandable members 406. When expanded, expandable members 406 increases a force applied by wearable component 106 on patient 102 (e.g., towards torso 103 of patient 102), thereby increasing contact forces between electrodes 210 and skin of patient 102 (e.g., to improve sensing capabilities of electrodes 210).

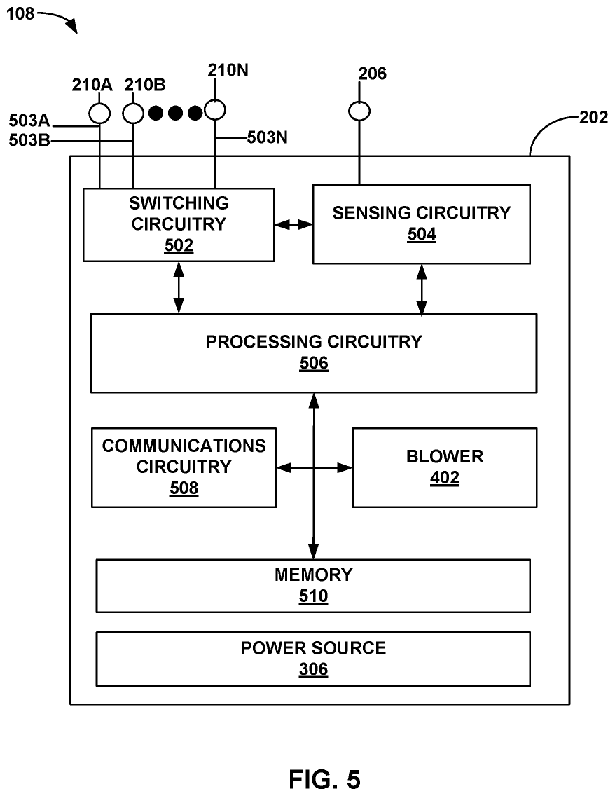

FIG. 5 is a functional block diagram illustrating an example configuration of computing device 108 of FIG. 1. In the example illustrated in FIG. 5, computing device 108 includes switching circuitry 502, sensing circuitry 504, processing circuitry 506, communications circuitry 508, blower 402, memory 510, and power source 306. In some examples, computing device 108 may not include blower 402 and/or may be electrically connected to blower 402 disposed in wearable component 106. The various circuitry may be, or include, programmable or fixed function circuitry configured to perform the functions attributed to respective circuitry. Memory 510 may store computer-readable instructions that, when executed by processing circuitry 506, cause computing device 108 to perform various functions. Memory 510 may be a storage device or other non-transitory medium. The components of computing device 108 illustrated in FIG. 5 may be housing within a housing of computing device 108 (e.g., as formed by front housing 302 and rear housing 313).

Switching circuitry 502 is coupled to electrodes 210A-210N via conductors 503A-503N (collectively referred to as “conductors 503”). Switching circuitry 502 may include one or more switch arrays, one or more multiplexers, one or more switches (e.g., a switch matrix of other collection of switches), one or more transistors, or other electrical circuitry. Switching circuitry 502 is configured to direct electrical signals from electrodes 210 to sensing circuitry 504, e.g., to sense electrical signals from heart of patient 102 via selected combinations of electrodes 210. In some examples, switching circuitry 502 may be further coupled to additional sensors (e.g., strain gauge(s) 206, capacitors) on wearable component 106 via one or more of conductors 502. Switching circuitry 502 may be configured to direct sensed signals from the additional sensors to sensing circuitry 504, e.g., to determine one or more physiological signals (e.g., a respiration signal) and/or physiological parameters (e.g., a respiration rate) of patient 102 via the additional sensors on wearable component 106. The physiological signals and/or physiological parameters may include but is not limited to, a respiration signal of patient 102. In some examples, one or more of the additional sensors may be defined by two or more of electrodes 210 and switching circuitry 520 may direct sensed signals from the two or more electrodes 210 to sensing circuitry 504 to determine the physiological signals and/or physiological parameters.

Sensing circuitry 504 may include filters, amplifiers (e.g., sense amplifiers), analog-to-digital converters, capacitors, or other circuitry configured to sense electrical signals and convert the sensed electrical signals to ECG signals, physiological signals and/or physiological parameters via electrodes 210 and/or additional sensors (e.g., strain gauge(s) 206) on wearable component 106. In some examples, sensing circuitry 504 may include two or more sensing modules 320 (e.g., as illustrated in FIG. 3D), each of sensing modules 320 being configured to be coupled to one or more of electrodes 210 and/or the additional sensors. In some examples, sensing circuitry 504 may include a single sensing module 320 configured to be coupled to at least some of electrodes 210 and/or the additional sensors. Sensing circuitry 504 may sense and record ECG signals from electrodes 210 and/or sensors on wearable component 106. In some examples, ECG signals from electrodes 210 represent electrical activity of the heart of the patient 102 (e.g., depolarization of chamber(s) of the heart). In some examples, electrical signals from the additional sensors and/or electrodes 210 configured to be the additional sensors may correspond to physiological signals and/or physiological parameters of patient 102. Sensing circuitry 504 may store the physiological signals and/or physiological parameters in memory 510.

Processing circuitry 506 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 506 herein may be embodied as firmware, hardware, software or any combination thereof.

Processing circuitry 506 may determine that patient 102 is experiencing a cardiac condition (e.g., arrhythmia, tachycardia) based on the sensed ECG signals from the heart. Processing circuitry 506 may compare one or more characteristics of the sensed ECG signals against threshold conditions and determine that patient 102 is experiencing a cardiac condition based on a determination that at least one of the characteristics of the sensed ECG signals satisfies a threshold condition corresponding to the cardiac condition. The one or more characteristics of the sensed ECG signals may include, but are not limited to, an amplitude of the sensed ECG signals, a frequency of the sensed ECG signals, morphology of the sensed ECG signals, changes in the amplitude frequency, and/or morphology of the sensed ECG signals, rates of change in the amplitude, the frequency, and/or morphology of the sensed ECG signals, RR intervals (heart rates) or other intervals between waves of the ECG signal, variability of such intervals, morphologies of such waves or variability of morphologies, or any other characteristic capable of being used as an indicator to detect and/or predict one or more cardiac conditions. In some examples, based on a determination that patient 102 is experiencing, has experienced within a threshold period of time (e.g., days, weeks, months), or will experience a cardiac condition (e.g., an arrhythmia or heart failure event) within a threshold period of time (e.g., minutes, hours), processing circuitry 506 may cause communications circuitry 508 to transmit the sensed ECG signals to one or more of external device 110, network 112, and/or computing device(s) 114. In some examples, processing circuitry 506 may detect occurrences of arrhythmias or other episodes of cardiac conditions, store ECG and/or other data corresponding to the episode, and transmit the data to one or more of external device 110, network 112, and/or computing device(s) 114.

Processing circuitry 506 may determine, based on the sensed signals from electrodes 210 and/or the additional sensors, whether patient 102 is properly wearing wearable component 106. When patient 102 improperly wears wearable component 106, one or more of electrodes 210 may be misplaced on torso 103 of patient 102 and may cause sensing circuitry 504 to sense inaccurate, altered, and/or distorted ECG signals from the heart of patient 102. In some examples, wearable component 106 may be too loose around torso 103 and lead to improper contact between torso 103 and one or more electrodes of electrodes 210. The improper contact may also cause sensing circuitry 504 to sense inaccurate, altered, and/or distorted ECG signals. Processing circuitry 506 may determine, based on noise signals in the sensed signals (e.g., sensed ECG signals, sensed respiration signals) from wearable component 106, that wearable component 106 is improperly worn and may suspend sensing of ECG signals from patient 102, e.g., to maintain power duration and/or storage of computing device 108 and/or to increase accuracy of determinations of any cardiac conditions experienced by patient 102.

In some examples, processing circuitry 506 may determine, from the sensed signals, a respiration signal and/or an ECG signal of patient 102 and determine a noise signal in the respiration signal and/or the ECG signal. Processing circuitry 506 may then determine that wearable component 106 is improperly worn, e.g., based on a determination that one or more characteristics of the determined noise signal satisfies a threshold noise condition. The one or more characteristics may include, but are not limited to, an amplitude of the noise signal, a frequency of the noise signal, the amplitude of the noise signal relative to the amplitude of the respiration signal and/or the amplitude of the ECG signal, a rate of change and/or slope of the noise signal, a number of changes in the noise signal during a period of time, an amplitude of the noise signal at certain frequencies and/or ranges of frequencies, or the like. The threshold noise conditions may be predetermined and may be based on prior sensed information from patient 102 and/or prior sensed information from other patients.

Processing circuitry 506 may determine the respiration signal for patient 102 based on voltage outputted by strain gauge(s) 206 and/or changes in capacitance between two or more of electrodes 210. Processing circuitry 506 may convert the sensed voltage and/or the sensed capacitance to a respiration signal and corresponding respiration rate for patient 102. In some examples, other physiological signals and/or parameters may be used by processing circuitry 506 to determine the respiration signal.

In some examples, processing circuitry 506 may determine that wearable component 106 is improperly worn by patient 102 based on one of the noise signals of the respiration signal or the ECG signal. In some examples, processing circuitry 506 may determine that wearable component 106 is improperly worn based on the noise signal of one of the respiration signal or the ECG signal and validate the determination based on the noise signal of the other of the respiration signal or the ECG signal.

Based on a determination that wearable component 106 is improperly worn by patient 102, processing circuitry 506 may cause sensing circuitry 504 to suspend sensing of the ECG signals from the heart of patient 102. Processing circuitry 506 may suspend the sensing of the ECG signals to reduce power consumption of computing device 108 when wearable component 106 is improperly worn. In some examples, processing circuitry 506 may suspend the sensing of the ECG signals to prevent storage of inaccurate electrical signals in memory 510.

Processing circuitry 506 may temporarily or permanently suspend the sensing of the ECG signals. In some examples, processing circuitry 506 may cause sensing circuitry 504 to suspend the sensing of the ECG signals, e.g., for a threshold period of time, until processing circuitry 506 receives a user input (e.g., from external device 110, network 112, computing device(s) 114, until processing circuitry 506 determines that the noise signals no longer satisfies the threshold noise conditions, or the like. In some examples, processing circuitry 506 may power off and/or enter a suspended mode in response to determining that wearable component 106 is improperly own. In some examples, processing circuitry 506 may instruct signal generation circuitry of computing device 108 (not pictured) to transmit electrical signals to sensing circuitry 504 (e.g., to sense amplifiers of sensing circuitry 504) to saturate sensing circuitry 504, e.g., to prevent sensing of ECG signals of the heart via electrode 210. In some examples, sensing circuitry 504 may suspend the sensing of the ECG signals from the heart of patient 102 while continuing to sense signals from the one or more additional sensors of wearable component 106, e.g., to determine physiological signals and/or parameters of patient 102.

In some examples, based on the determination that wearable component 106 is improperly worn by patient 102, processing circuitry 506 may transmit instructions to blower 402 to cause blower 402 to expand at least some of expandable members 406, e.g., to increase contact between electrodes 210 and patient 102 and increase sensing sensitivity and/or accuracy of electrodes 210. In some examples, processing circuitry 506 may determine (e.g., based on the noise signals of sensed signals from a plurality of sensors disposed at different positions and/or orientations within wearable component 106), a position and/or orientation of wearable component 106 relative to patient 102 (e.g., relative to torso 103). Processing circuitry 506 may determine which expandable members 406 to expand based on the determined position and/or orientation of wearable component 106 and cause blower 402 to selectively expand one or more of expandable members 406 to increase sensing sensitivity and/or accuracy of some of electrodes 210. In some examples, processing circuitry 506 may case blower 402 to expand all of expandable members 406.

In some examples, processing circuitry 506 is configured to determine one or more activity states of patient 102 based on sensed signals (e.g., from electrodes 210, strain gauge(s) 206, and/or accelerometers disposed within ECM system 104). The sensed signals may include, but are not limited to, signals indicative of a heart rate of patient 102, signals indicative of a pulse rate of patient 102, respiration signals from patient 102, blood oxygen saturation of patient 102, and/or acceleration of one or more regions of patient 102 (e.g., as detected by accelerometers within ECM system 104). Processing circuitry 506 may determine, for each activity state, a type of the activity state and/or a duration of the activity state. Processing circuitry 506 may define a neuromorphic architecture. Processing circuitry 506 may retrieve instructions from memory 510 and apply a machine learning algorithm (e.g., via the neuromorphic architecture) based on the retrieved instructions to determine the one or more activity states.

Processing circuitry 506 may store the determined activity states of patient 102 in memory 510. Processing circuitry 506, external device 110, and/or computing device(s) 114 may determine trends and/or changes in activity states of patient 102 over time based on the determined activity states.

In some examples, based on the determination that the wearable component 106 is improperly worn by patient 102, processing circuitry 506 transmits a notification, via communications circuitry 508, to external device 110, network 112, and/or computing device(s) 114 indicating that wearable component 106 is improperly positions and prompting patient 102 and/or one or more other individuals to re-position wearable component 106 on patient 102.

Communications circuitry 508 (also referred to as “telemetry circuitry 508”) supports wireless communication between computing device 108 and external device 110 and/or network 112. Processing circuitry 506 may provide collected data (e.g., sensed ECG signals, respiration signal of patient 102, respiration rate of patient 102) to external device 110, network 112, and/or computing device(s) 114 via communications circuitry 508. Communications circuitry 508 may receive user inputs from external device 110 and/or network 112 and transmit the received user inputs to processing circuitry 506. Communications circuitry 508 may accomplish communication by radiofrequency (RF) communications techniques, e.g., via an antenna (not shown). Communications circuitry 508 may include a radio transceiver configured for communication according to standards or protocols, such as 3G, 4G, 5G, Wi-Fi (e.g., 802.11 or 802.15 ZigBee), Bluetooth®, or Bluetooth® Low Energy (BLE).

In some examples, the components of computing device 108 may include computing components of a Reveal LINQ™ or LINQ II™ cardiac monitor available from Medtronic Plc., Dublin, Ireland. In such examples, computing device 108 may be capable of communicating with network 112 such as the CareLink™ Network available from Medtronic Plc., Dublin, Ireland, e.g., to detect cardiac conditions, store sensed signals, and transmit sensed signals and/or information on detected cardiac conditions across multiple computing devices.

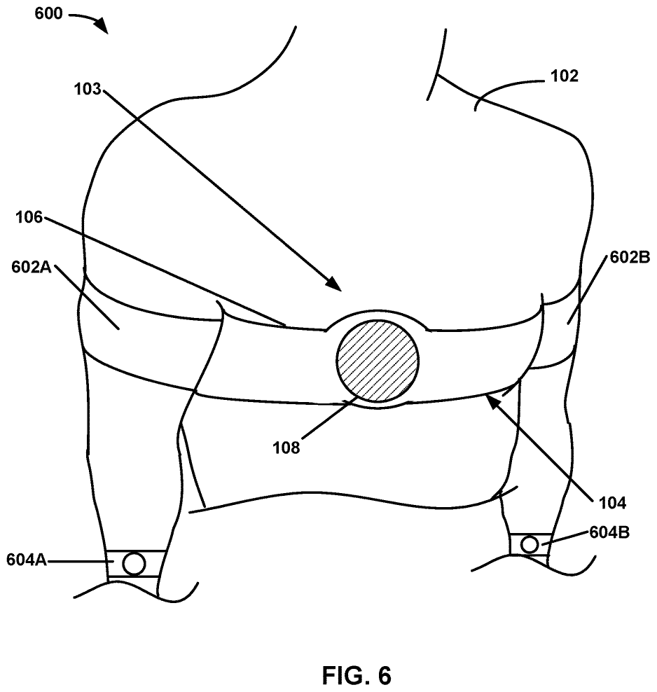

FIG. 6 is a conceptual diagram illustrating an example medical device system 600 including ECM system 104 and one or more external monitoring devices. As illustrated in FIG. 6, and described previously herein, ECM system 104 is worn around torso 103 of patient 102. The one or more external monitoring devices may be in communication with computing device 108 of ECM system 104 and may transmit information sensed by the one or more external monitoring devices to ECM system 104. The one or more external monitoring devices may include, but are not limited to, wearable sleeves 602A-602B (collectively referred to as “wearable sleeves 602”) or wrist monitors 604A-604B (collectively referred to as “wrist monitors 604”). While the one or more external monitoring devices are described below primarily with reference to wearable sleeves 602 and wrist monitors 604, the example processes described herein may be applied to other wearable monitoring devices.

Wearable sleeves 602 may be worn around an arm of patient 102 and may sense one or more signals from patient 102. Wearable sleeves 602 may be formed from a flexible material (e.g., flexible material 204). In some examples, flexible material 204 is an electrically conductive fabric defining one or more electrodes configured to sense signals from the arm of patient 102. In some examples, each of wearable sleeves 602 include one or more electrodes disposed within flexible material 204. Each of wearable sleeves 602 may include additional sensors (e.g., strain gauges, accelerometers) configured to sense signals from the arm of patient 102, e.g., to measure bioelectric impedance (e.g., to determine an occurrence of fluid congestion (e.g., edema)), to monitor blood pressure of patient 102, or the like. Patient 102 may wear one or more wearable sleeves 602 around each arm, e.g., at one or more locations along the arm.

Wrist monitors 604 may be worn around the wrists of patient 102. Each of wrist monitors 604 may sense one or more signals from patient 102 including, but is not limited to, an oxygen saturation of patient 102. In some examples wrist monitors 604 may be wrist-worn plethysmography devices.

Each of wearable sleeves 602 and wrist monitors 604 may be in wired or wireless communication with ECM system 104 (e.g., with computing device 108). In some examples, each of wearable sleeves 602 and wrist monitors 604 includes power sources (e.g., removable and/or rechargeable power sources) and computing circuitry configured to sense electrical signals from patient 102. ECM system 104 may determine whether patient 102 is experiencing cardiac conditions and/or other health conditions based at least in part on the sensed signals from wearable sleeves 602 and wrist monitors 604.

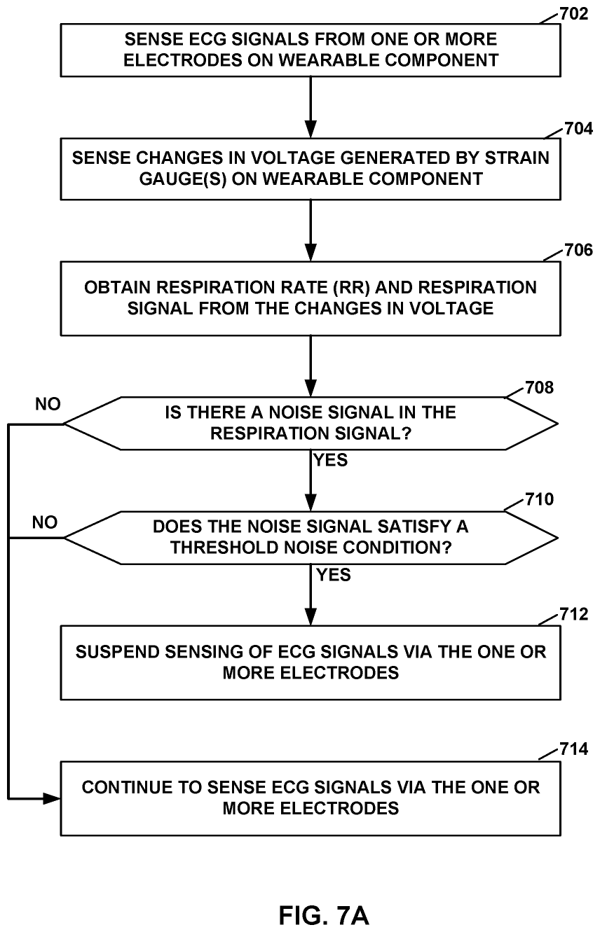

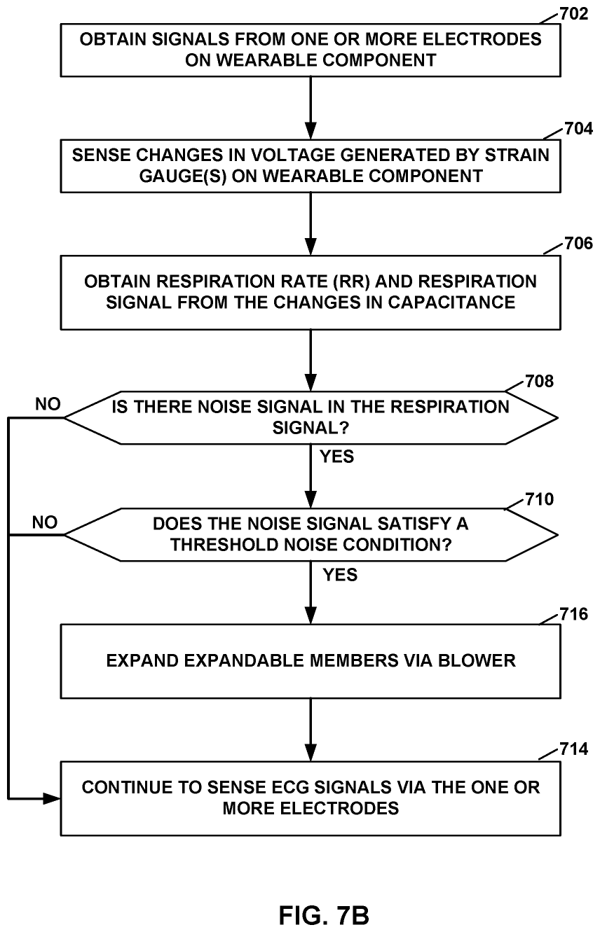

FIG. 7A is a flow diagram illustrating an example method of sensing signals from a patient using an example medical device system 100. While the example method illustrated in FIG. 7A is primarily described as sensing ECG signals from patient 102, system 100 may apply the example method described herein to sense other electrical signals from the heart of patient 102. While FIG. 7A illustrated use of the voltage from strain gauge(s) 206 to determine the respiration signal of patient 102, other example sensed signals from patient 102 (e.g., changes in capacitance from two or more of electrodes 210 configured to be capacitors and/or two or more capacitors) may be used to determine the respiration signal.

Computing device 108 of ECM system 104 may sense ECG signals from one or more electrodes 210 on wearable component 106 (702). Electrodes 210 may be disposed on rear surface 214 of wearable component 106 or defined by an electrically conductive (e.g., dry-electrode) flexible material 204. Computing device 108 may be removably secured to wearable component 106 (e.g., within recess 202 of wearable component 106) and configured to be electrically connected to electrodes 210. Computing device 108 senses the ECG signals from patient 102 via electrodes 210. The ECG signals correspond to electrical activity of the heart of patient 102. Computing device 108 may store the sensed ECG signals in memory 510 of computing device 108. Computing device 108 may transmit the sensed ECG signals to external device 110, network 112, and/or computing device(s) 114 of system 100.

In some examples, computing device 108 determines whether patient 102 is experiencing, has experienced, or will experience a cardiac condition based on the sensed ECG signals. Computing device 108 may determine whether one or more characteristics (e.g., amplitude, frequency, rate of change) of the sensed ECG signals satisfies a threshold condition and determine that patient 102 is experiencing, has experienced, or will experience the cardiac condition based on the satisfaction of the threshold condition. In some examples, computing device 108 may transmit the sensed ECG signals to external device 110, network 112, and/or computing device(s) 114 based on satisfaction of the threshold condition.

Computing device 108 may sense changes in voltage generated by strain gauge(s) 206 on wearable component 106 (704) as torso 103 of patient 102 expands and contracts in response to respiration of patient 102, a strain or stress is applied to strain gauge(s) 206 disposed in wearable component 106 and along longitudinal axis 205 of wearable component 106. The applied strain or stress causes strain gauge(s) 206 to elongated or compress and generate a voltage. Computing device 108 may be electrically connected to strain gauge(s) 206 and may sense the changes in voltage generated by strain gauge(s) 206. Computing device 108 may obtain a respiration rate (RR) and respiration signal from the changes in voltage (706). Computing device 108 may filter and/or convert the sensed change in voltage to a respiration signal. Computing device 108 may determine, based on the respiration signal over a set period of time, a respiration rate of patient 102. In some examples, computing device 108 may output the determined RR and/or respiration signal to external device 110, network 112, and/or computing device(s) 114.

Computing device 108 may determine whether there is a noise signal in the respiration signal (708). Based on a determination that there is no noise signal in the respiration signal (“NO” branch of 708), Computing device 108 may continue to sense ECG signals via one or more of electrodes 210 (714).

Based on a determination that there is a noise signal in the respiration signal (“YES” branch of 708), computing device 108 may determine whether the noise signal satisfies a threshold noise condition (710). Computing device 108 may determine whether the noise signal satisfies the threshold noise condition by determining whether one or more characteristics of the noise signal satisfies the threshold noise condition. The one or more characteristics may include, but is not limited to, an amplitude of the noise signal, a frequency of the noise signal, the amplitude of the noise signal relative to the amplitude of the respiration signal and/or the amplitude of the ECG signal, a rate of change and/or slope of the noise signal, a number of changes in the noise signal during a period of time, an amplitude of the noise signal at certain frequencies and/or ranges of frequencies, or the like.

Based on a determination that the noise signal does not satisfies the threshold noise condition (“NO” branch of 710), computing device 108 may continue to sense ECG signals via one or more of electrodes 210 (714). Based on a determination that the noise signal satisfies the threshold noise condition (“YES” branch of 710), computing device 108 suspend sensing of ECG signals via one or more of electrodes 210 (712).

Computing device 108 may suspend sensing of ECG signals, e.g., to reduce power consumption, prevent storage of inaccurate ECG signals, and/or increase the accuracy of the determination of cardiac conditions experienced by patient 102. Computing device 108 may suspend the sensing of ECG signals by saturating one or more sense amplifiers of computing device 108. In some examples, computing device 108 may power off to suspend the sensing of ECG signals. Computing device 108 may temporarily suspend the sensing of ECG signals. In some examples, computing device 108 may resume the sensing of ECG signals based on reception of user input, after passage of a predetermined period of time, and/or based on a determination by computing device 108 that the noise signal of the respiration signal no longer satisfies the threshold noise condition.

FIG. 7B is a flow diagram illustrating an example method of expanding expandable members 406 of an example medical device system 100. Computing device 108 of ECM system 104 may perform steps 702-710 and 714 in accordance with the example method described with respect to FIG. 7A.

As illustrated in FIG. 7B, based on a determination that the noise signal satisfies the threshold noise condition (“YES” step of 710), computing device 108 may expand expandable members 406 via blower 402 (e.g., within computing device 108). Expandable members 406 are disposed within wearable component 106 and/or defined by flexible material 204 of wearable component 106. Expansion of expandable members 406 may increase a contact force between electrodes 210 and skin of patient 102, e.g., to increase sensing sensitivity and/or sensing accuracy of electrodes 210. Blower 402 may output air into expandable members 406 via channels 404 to expand expandable members 406 into expanded configurations. In some examples, blower 402 may continue to output air into expandable members 406 to maintain expandable members 406 in the expanded configurations.

Computing device 108 may continue to sense ECG signals via the one or more electrodes (714). In some examples, computing device 108 may continue to sense ECG signals for a set period of time and determine whether expansion of expandable members 406 reduce the noise signals of respiration signal such that the noise signal no longer satisfies the threshold noise condition (e.g., characteristics of the noise signal no longer satisfies the threshold condition). Based on a determination that the noise signal continues to satisfy the threshold condition, computing device 108 may suspend the sensing of the ECG signals, e.g., in accordance with step 712 of the example method of FIG. 6A.

In some examples, computing device 108 may expand expandable members 406 via blower 402 based on a determination that the noise signal satisfies the threshold noise condition by a threshold amount (e.g., corresponding to a mild loss of contact). In such examples, expansion of expandable members 406 increases contact between electrodes 210 and skin of patient 102 and alleviates the mild loss of contact. In some examples, computing device 108 may determine that the noise signal exceeds the threshold noise condition by the threshold amount, e.g., corresponding to a gross loss of contact. In such examples, expansion of expandable members 406 does not alleviate the gross loss of contact and computing device 108 proceeds directly to suspension of the sensing of the ECG signals without expanding expandable members 406.

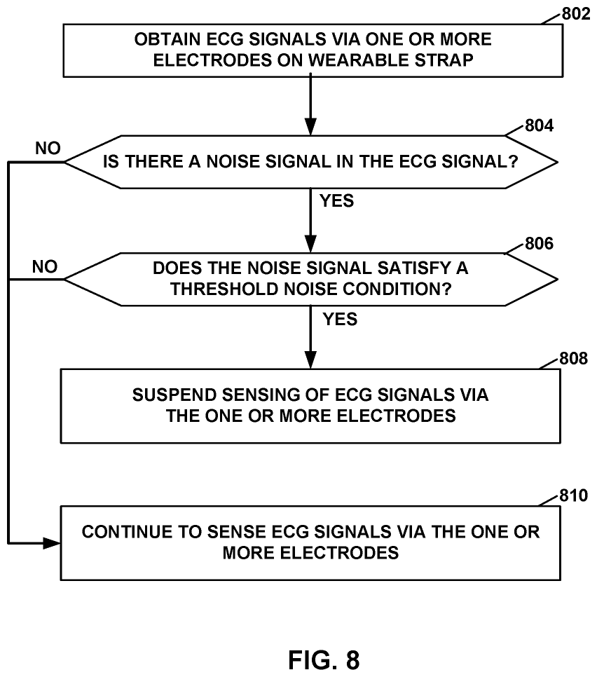

FIG. 8 is a flow diagram illustrating another example method of sensing electrical signals from patient 102 using an example medical device system 100. System 100 may apply the example method of FIG. 8 to sense electrical signals from the heart of patient 102 and/or to determine whether wearable component 106 of ECM system 104 of system 100 is properly worn by patient 102.

Computing device 108 may obtain ECG signals of patient 102 via one or more electrodes 210 on wearable component 106 (802). Electrodes 210 are placed in contact with patient 102 and electrically connected to computing device 108 (e.g., via an electrically conductive (e.g., dry-electrode) flexible material 204). Electrodes 210 may be configured to detect signals corresponding to electrical activity of the heart of patient 102 and filter the obtained signal to determine an ECG signal. The ECG signal corresponds to electrical activity of the heart and may be used by computing device 108 to determine whether patient 102 is experiencing, has experienced, and/or will experience a cardiac condition. Computing device 108 may store the sensed ECG signals and/or transmit the ECG signals to external device 110, network 112, and/or computing device(s) 114. In some examples, computing device 108 may determine, based on the sensed ECG signals, whether patient 102 has experienced, is experiencing, or will experience a cardiac condition, e.g., according to the example methods described above.

Computing device 108 may determine whether there is a noise signal in the ECG signal (804). Based on a determination that there is no noise signal in the ECG signal (“NO” branch of 804), computing device 108 may continue to sense ECG signals via the one or more electrodes 210 (810). Based on a determination that there is a noise signal in the ECG signal (“YES” branch of 804), computing device 108 determines whether the noise signal in the ECG signal satisfies a threshold noise condition (806).

Computing device 108 may determine whether the noise signal in the ECG signal satisfies one or more threshold noise conditions, e.g., in accordance with one or more example methods as described above. Based on a determination that the noise signal does not satisfy the threshold noise condition (“NO” branch of 806), computing device 108 may continue to sense ECG signals via one or more electrodes 210 (810). Based on a determination that the noise signal does satisfy the threshold noise condition (“YES” branch of 806), computing device 108 may suspend the sensing of ECG signals via the one or more electrodes 210 (808). In some examples, based on the determination that the noise signal satisfies the threshold noise condition, computing device 108 may expand expandable members 406, e.g., in accordance with the example method described in FIG. 6B.

In some examples, medical device system 100 may determine whether wearable component 106 is properly worn by patient 102 by applying the example method illustrated in either FIG. 7A or FIG. 8. In some examples medical device system 100 may determine whether wearable component 106 is properly worn by applying the example methods of FIGS. 7A and 8 and/or apply the example method of one of FIGS. 7A and 8 to validate determinations made by applying the example method of the other of FIGS. 7A and 8.

The devices, systems, and techniques of this disclosure provides improvements over other medical therapy delivery systems. The establishment of a connection between external devices and implantable devices prior to deliver of medical therapies ensures availability of external aid in case of an unintended event occurrence and thereby reduces the severity of impact of any such unintended events. In some examples, using context factors to select medical therapies based on the aggressiveness of each medical therapy may increase the accuracy of tachyarrhythmia prediction, reduce the severity of impact of unintended event occurrences, and/or increase efficacy of the medical therapies while reducing risk of an unintended event.

The techniques of this disclosure may be implemented in a wide variety of computing devices, medical devices, or any combination thereof. Any of the described units, modules, or components may be implemented together or separately as discrete but interoperable logic devices. Depiction of different features as modules or units is intended to highlight different functional aspects and does not necessarily imply that such modules of units must be realized by separate hardware or software components. Rather, functionality associated with one or more modules or units may be performed by separate hardware or software components, or integrated within common or separate hardware or software components.

The disclosure contemplates computer-readable storage media comprising instructions to cause a processor to perform any of the functions and techniques describes herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various modules and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. For example, any module described herein may include electrical circuitry configured to perform the features attributed to that particular module, such as fixed function processing circuitry, programmable processing circuitry, or combinations thereof.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

It should be noted that medical device system 100, and the techniques described herein, may not be limited to use in a human patient. In alternative examples, medical device system 100 may be implemented in non-human patients, e.g., primates, canines, equines, pigs, felines, ursids, pachyderms, and spheniscids. These other animals may undergo clinical or research therapies that my benefit from the subject matter of this disclosure. Various examples are described herein, such as the following examples.

Example 1: a medical device system comprising: a wearable component configured to encircle a portion of a torso of a patient, the wearable component defining a longitudinal axis and comprising: an electrically conductive fabric; a plurality of electrically active regions defined by the electrically conductive fabric and disposed along the longitudinal axis of the electrically conductive fabric, each of the plurality of electrically active regions being configured to contact skin of the patient and sense an electrocardiogram (ECG) signal of a heart of the patient; one or more strain gauges disposed within the electrically conductive fabric and along the longitudinal axis; and a recess; and a computing module disposed within the recess, the computing module comprising: sensing circuitry electrically connected to the plurality of electrically active regions and the one or more strain gauges; and processing circuitry configured to: cause the sensing circuitry to sense the ECG signal via the plurality of electrically active regions and measure voltage values from the one or more strain gauges over time; determine, based on the voltage values, a respiration signal of the patient and a noise signal in the respiration signal; determine whether the noise signal in the respiration signal satisfies a threshold condition; and based on a determination that the noise signal satisfies the threshold condition, cause the sensing circuitry to suspend sensing the ECG signal.

Example 2: the medical device system of example 1, wherein the sensing circuitry comprises one or more sense amplifiers, and wherein to cause the sensing circuitry to suspend sensing the ECG signal, the processing circuitry is configured to transmit a signal to the sensing circuitry to saturate the one or more sense amplifiers.

Example 3: the medical device system of example 1, wherein to cause the sensing circuitry to suspend sensing the ECG signal, the processing circuitry is configured to power off the computing module in response to the determination that the noise signal satisfies the threshold condition.

Example 4: the medical device system of any of examples 1-3, wherein the processing circuitry is configured to cause the sensing circuitry to suspend sensing the ECG signal for a predetermined period of time.

Example 5: the medical device system of any of examples 1-4, wherein the processing circuitry is configured to cause the sensing circuitry to suspend sensing the ECG signal until the processing circuitry determines that the noise signal in the respiration signal no longer satisfies the threshold condition.

Example 6: the medical device system of any of examples 1-5, wherein the wearable component comprises one or more expandable members in fluid communication with the recess, and wherein the computing module further comprises: a blower configured to expand the one or more expandable members, wherein when expanded, the one or more expandable members are configured to increase a contact force between the skin and at least one of the plurality of electrically active regions.

Example 7: the medical device system of example 6, wherein the processing circuitry is further configured to: based on the determination that the noise signal satisfies the threshold condition, cause the blower to expand the one or more expandable members.

Example 8: the medical device system of any of examples 1-7, wherein the computing module is removably secured within the recess.

Example 9: the medical device system of example 8, wherein the computing module is removably secured within the recess via a fixation mechanism, and wherein the sensing circuitry of the computing module is electrically connected to the electrically active regions and the one or more strain gauges through the fixation mechanism.

Example 10: the medical device system of example 9, wherein the fixation mechanism comprises a plurality of spring clips.

Example 11: the medical devices system of any of examples 1-10, wherein the electrically conductive fabric comprises a dry electrode material defining the plurality of electrically active regions.

Example 12: the medical device system of any of examples 1-11, wherein the computing module further comprises a removable power source.

Example 13: the medical device system of any of examples 1-12, wherein the threshold condition comprises a threshold noise signal amplitude, and wherein the processing circuitry is configured to determine that the noise signal satisfies the threshold condition based on a determination that an amplitude of the noise signal is greater than or equal to the threshold noise signal amplitude.

Example 14: the medical device system of any of examples 1-13, wherein the noise signal satisfies the threshold condition when the wearable component is not in a predetermined position around the torso of the patient.

Example 15: the medical device system of example 14, wherein the computing module comprises communications circuitry, and wherein the processing circuitry is configured to: transmit, via the communications circuitry, the sensed ECG signals to one or more of an external computing device or a computing network.

Example 16: the medical device system of example 15, wherein the processing circuitry is configured to: determine, based on the sensed ECG signals, whether the patient is experiencing an arrhythmia; and based on a determination that the patient is experiencing the arrhythmia, transmit, via the communications circuitry, the sensed ECG signals to the one or more of the external computing device or the computing network.

Example 17: a medical device system comprising: a wearable component configured to encircle a portion of a torso of a patient, the wearable component defining a longitudinal axis and comprising: an electrically conductive fabric; a plurality of electrically action regions defined by the electrically conductive fabric and disposed along the longitudinal axis of the electrically conductive fabric, each of the plurality of electrically active regions being configured to contact skin of the patient and sense an electrocardiogram (ECG) signal of a heart of the patient; and a recess; and a computing module disposed within the recess, the computing module comprising: sensing circuitry electrically connected to the plurality of electrically active regions; and processing circuitry configured to: cause the sensing circuitry to sense the ECG signal via the plurality of electrically active regions; determine a noise signal within the sensed ECG signal; determine whether the noise signal satisfies a threshold condition; and based on a determination that the noise signal satisfies the threshold condition, cause the sensing circuitry to suspend sensing the ECG signal.

Example 18: the medical device system of example 17, wherein the sensing circuitry comprises one or more sense amplifiers, and wherein to cause the sensing circuitry to suspend sensing the ECG signal, the processing circuitry is configured to transmit a signal to the sensing circuitry to saturate the one or more sense amplifiers.

Example 19: the medical device system of example 17, wherein to cause the sensing circuitry to suspend sensing the ECG signal, the processing circuitry is configured to power off the computing module in response to the determination that the noise signal satisfies the threshold condition.

Example 20: the medical device system of any of examples 17-19, wherein the processing circuitry is configured to cause the sensing circuitry to suspend sensing the ECG signal for a predetermined period of time.

Example 21: the medical device system of any of examples 17-20, wherein the processing circuitry is configured to cause the sensing circuitry to suspend sensing the ECG signal until the processing circuitry determines that the noise signal no longer satisfies the threshold condition.

Example 22: the medical device system of any of examples 17-21, wherein the wearable component comprises one or more expandable member sin fluid communication with the recess, and wherein the computing module further comprises: a blower configured to expand the one or more expandable members, wherein when expanded, the one or more expandable members are configured to increase a contact force between the skin and at least one of the plurality of electrically active regions.

Example 23: the medical device system of example 22, wherein the processing circuitry is further configured to: based on the determination that the noise signal satisfies the threshold condition, cause the blower to expand the one or more expandable members.

Example 24: the medical device system of any of examples 17-23, wherein the computing module is removable secured within the recess.

Example 25: the medical device system of example 24, wherein the computing module is removably secured within the recess via a fixation mechanism, and wherein the sensing circuitry of the computing module is electrically connected to the electrically active regions through the fixation mechanism.

Example 26: the medical device system of example 25, wherein the fixation mechanism comprises a plurality of spring clips.

Example 27: the medical device system of any of examples 17-26, wherein the electrically conductive fabric comprises a dry electrode material defining the plurality of electrically active regions.

Example 28: the medical device system of any of examples 17-27, wherein the computing module further comprises a removable power source.

Example 29: the medical device system of any of examples 17-28, wherein the threshold condition comprises a threshold noise signal amplitude, and wherein the processing circuitry is configured to determine that the noise signal satisfies the threshold condition based on a determination that an amplitude of the noise signal is greater than or equal to the threshold noise signal amplitude.

Example 30: the medical device system of any of examples 17-29, wherein the noise signal satisfies the threshold condition when the wearable component is not in a predetermined position around the torso of the patient.

Example 31: the medical device system of example 30, wherein the computing module comprises communications circuitry, and wherein the processing circuitry is configured to: transmit, via the communications circuitry, the sensed ECG signals to one or more of an external computing device or a computing network.

Example 32: the medical device system of example 31, wherein the processing circuitry is configured to: determine, based on the sensed ECG signals, whether the patient is experiencing an arrhythmia; and based on a determination that the patient is experiencing the arrhythmia, transmit, via the communications circuitry, the sensed ECG signals to the one or more of the external computing device or the computing network.

Example 33: a computing device configured to sense an electrocardiogram (ECG) signal from a patient, the computing device comprising: sensing circuitry comprising one or more sense amplifiers; processing circuitry configured to: sense, via the sensing circuitry, a signal from one or more sensors on a wearable component in contact with skin of the patient; determine a noise signal within the sensed signal; determine whether the noise signal satisfies a threshold condition; determine, based on a determination that the noise signal satisfies the threshold condition, that the patient is improperly wearing the wearable strap; and based on a determination that the patient is improperly wearing the wearable component, cause the sensing circuitry to suspend sensing the ECG signal; and a fixation mechanism configured to electrically connect the computing device the one or more sensors.

Example 34: the computing device of example 33, wherein the one or more sensors comprises one or more strain gauges, wherein the sensing circuitry is configured to measure voltage values over time via the one or more strain gauges, and wherein the processing circuitry is further configured to: determine, based on the measured voltage values, a respiration signal of the patient, wherein the sensed signal comprises the respiration signal, and wherein the noise signal of the sensed signal comprises a noise signal of the respiration signal.

Example 35: the computing device of example 34, wherein the threshold condition comprises a threshold noise signal amplitude, and wherein to determine that the noise signal satisfies the threshold condition, the processing circuitry is configured to determine that an amplitude of the noise signal of the respiration signal is greater than or equal to the threshold noise signal amplitude.

Example 36: the computing device of any of examples 33-35, wherein the one or more sensors comprises one or more electrically active regions disposed along the wearable component.

Example 37: the computing device of example 36, wherein the processing circuitry is configured to sense, via the sensing circuitry, the ECG signal from the one or more electrically active regions, wherein the sensed signal comprises the ECG signal, and wherein the noise signal of the sensed signal comprises a noise signal of the ECG signal.

Example 38: the computing device of example 37, wherein the threshold condition comprises a threshold noise signal amplitude, and wherein to determine that the noise signal satisfies the threshold condition, the processing circuitry is configured to determine that an amplitude of the noise signal of the ECG signal is greater than or equal to the threshold noise signal amplitude.

Example 39: the computing device of any of examples 36-38, wherein each of the one or more sense amplifiers is electrically connected to a corresponding electrically active region of the plurality of electrically active regions, and wherein each sense simplifier of the one or more sensed amplifiers is configured to sense the ECG signal from the skin of the patient.

Example 40: the computing device of any of examples 33-39, further comprising a blower configured to connect to one or more expandable members disposed on the wearable component, and wherein the processing circuitry is further configured to: based on the determination that the patient is improperly wearing the wearable component, engage the blower to expand the one or more expandable members to increase a contact force between the one or more sensors and the patient.

Example 41: the computing device of any of examples 33-40, wherein the fixation mechanism comprises one or more spring clips.

Example 42: the computing device of any of examples 33-41, further comprising a removable power source.

Example 43: a method comprising: sensing, by sensing circuitry of a computing module and via a plurality of electrically active regions disposed on a wearable component worn by a patient and configured to contact skin of the patient, an electrocardiogram (ECG) signal of a heart of the patient, wherein the wearable component is configured to encircle a portion of a torso of the patient, and wherein the wearable component comprises: an electrically conductive fabric defining the plurality of electrically active regions along a longitudinal axis of the wearable component; and a recess configured to retain the computing module; determining, by processing circuitry of the computing module and based on the ECG signal, a noise signal within the ECG signal; determining, by the processing circuitry, whether the noise signal satisfies a threshold condition; determining, by the processing circuitry and based on a determination that the noise signal satisfies the threshold condition, that the patient is improperly wearing the wearable component; and based on a determination that the patient is improperly wearing the wearable component, causing, by the processing circuitry, the sensing circuitry to suspend sensing the ECG signal.

Example 44: the method of example 43, wherein determining that the noise signal satisfies the threshold condition comprises: determining, by the processing circuitry, that a characteristic of the noise signal is greater than or equal to a threshold value corresponding to the characteristic.

Example 45: the method of any of examples 43 and 44, wherein the sensing circuitry comprises a plurality of sense amplifiers, wherein each sense amplifier of the plurality of sense amplifiers is electrically connected to a corresponding electrically active region of the plurality of electrically active regions, and wherein causing the sensing circuitry to suspend sensing the ECG signal comprises: transmitting a signal to the sensing circuitry to saturate the one or more sense amplifiers.

Example 46: the method of any of examples 43-45, further comprising: determining, by the processing circuitry, an amount of time since suspension of the sensing the ECG signal by the sensing circuitry; and based on a determination that the amount of time is greater than or equal to a predetermined period of time, causing, by the processing circuitry, the sensing circuitry to resume sensing the ECG signal.

Example 47: the method of any of examples 43-46, further comprising: determining, by the processing circuitry, whether the noise signal satisfies the threshold condition after suspension of the sensing of the ECG signal; and based on a determination that the noise signal no longer satisfies the threshold condition, causing, by the processing circuitry, the sensing circuitry to resume sensing the ECG signal.

Example 48: the method of any of examples 43-47, wherein the wearable component further comprises one or more strain gauges disposed along the longitudinal axis of the wearable component, and wherein the method further comprises: sensing, by the sensing circuitry and via the one or more strain gauges, measured voltage values over time; determining, by the processing circuitry and based on the measured voltage values, a respiration signal of the patient; determining, by the processing circuitry, a noise signal of the respiration signal; determining, by the processing circuitry, whether the noise signal of the respiration signal satisfies a threshold condition of the noise signal; and based on a determination that the noises signal of the respiration signal satisfies a threshold condition of the noise signal, causing, by the processing circuitry, the sensing circuitry to suspend sensing the ECG signal.

Example 49: the method of any of examples 43-48, wherein causing the sensing circuitry to suspend sensing the signal comprises: powering off, by the processing circuitry, the computing module in response to the determination that the noise signal satisfies the threshold condition.

Example 50: the method of any of examples 43-49, wherein the wearable component comprises one or more expandable members in fluid communication with the recess and wherein when expanded the one or more expandable members are configured to increase a contact force between the skin and at least one of the plurality of electrically active regions.

Example 51: the method of example 50, further comprising: based on the determination that the noise signal satisfies the threshold condition, causing, by the processing circuitry, a blower disposed within the computing module to expand the one or more expandable members.

Example 52: the method of any of examples 43-51, wherein the computing module is retained within the recess by a fixation mechanism.

Example 53: the method of example 52, wherein the fixation mechanism comprises a plurality of spring clips.

Example 54: the method of any of examples 43-53, further comprising: based on the determination that the patient is improperly wearing the wearable component transmitting, by the processing circuitry and via communications circuitry of the computing module, a notification to one or more of an external computing device or a computing network that the patient is improperly wearing the wearable component.

Various examples of the disclosure have been described. Any combination of the described systems, operations, or functions is contemplated. These and other examples are within the scope of the following claims.

1. A medical device system comprising:
a wearable component configured to encircle a portion of a torso of a patient, the wearable component defining a longitudinal axis and comprising:
an electrically conductive fabric;
a plurality of electrically active regions defined by the electrically conductive fabric and disposed along the longitudinal axis of the electrically conductive fabric, each of the plurality of electrically active regions being configured to contact skin of the patient and sense an electrocardiogram (ECG) signal of a heart of the patient;
one or more strain gauges disposed within the electrically conductive fabric and along the longitudinal axis; and
a recess; and
a computing module disposed within the recess, the computing module comprising:
sensing circuitry electrically connected to the plurality of electrically active regions and the one or more strain gauges; and
processing circuitry configured to:
cause the sensing circuitry to sense the ECG signal via the plurality of electrically active regions and measure voltage values from the one or more strain gauges over time;
determine, based on the voltage values, a respiration signal of the patient and a noise signal in the respiration signal;
determine whether the noise signal in the respiration signal satisfies a threshold condition; and
based on a determination that the noise signal satisfies the threshold condition, cause the sensing circuitry to suspend sensing the ECG signal.

2. The medical device system of claim 1, wherein the sensing circuitry comprises one or more sense amplifiers, and wherein to cause the sensing circuitry to suspend sensing the ECG signal, the processing circuitry is configured to transmit a signal to the sensing circuitry to saturate the one or more sense amplifiers.

3. The medical device system of claim 1, wherein to cause the sensing circuitry to suspend sensing the ECG signal, the processing circuitry is configured to power off the computing module in response to the determination that the noise signal satisfies the threshold condition.

4. (canceled)

5. The medical device system of claim 1, wherein the processing circuitry is configured to cause the sensing circuitry to suspend sensing the ECG signal until the processing circuitry determines that the noise signal in the respiration signal no longer satisfies the threshold condition.

6. The medical device system of claim 1, wherein the wearable component comprises one or more expandable members in fluid communication with the recess, and wherein the computing module further comprises:
a blower configured to expand the one or more expandable members, wherein when expanded, the one or more expandable members are configured to increase a contact force between the skin and at least one electrically active region of the plurality of electrically active regions.

7. The medical device system of claim 6, wherein the processing circuitry is further configured to:
based on the determination that the noise signal satisfies the threshold condition, cause the blower to expand the one or more expandable members.

8. The medical device system of claim 1, wherein the computing module is removably secured within the recess via a fixation mechanism, and wherein the sensing circuitry of the computing module is electrically connected to the electrically active regions and the one or more strain gauges through the fixation mechanism.

9. (canceled)

10. The medical device system of claim 1, wherein the electrically conductive fabric comprises a dry defining the plurality of electrically active regions.

11. (canceled)

12. The medical device system of claim 1, wherein the threshold condition comprises a threshold noise signal amplitude, and wherein the processing circuitry is configured to determine that the noise signal satisfies the threshold condition based on a determination that an amplitude of the noise signal is greater than or equal to the threshold noise signal amplitude.

13. The medical device system of claim 1, wherein the noise signal satisfies the threshold condition when the wearable component is not in a predetermined position around the torso of the patient.

14. (canceled)

15. The medical device system of claim 1, wherein the computing module further comprises communications circuitry, and wherein the processing circuitry is configured to:
determine, based on the sensed ECG signals, whether the patient is experiencing an arrhythmia; and
based on a determination that the patient is experiencing the arrhythmia, transmit, via the communications circuitry, the sensed ECG signals to one or more of an external computing device or a computing network.

16. A computing device configured to sense an electrocardiogram (ECG) signal from a patient, the computing device comprising:
sensing circuitry comprising one or more sense amplifiers;
processing circuitry configured to:
sense, via the sensing circuitry, a signal from one or more sensors on a wearable component in contact with skin of the patient;
determine a noise signal within the sensed signal;
determine whether the noise signal satisfies a threshold condition;
determine, based on a determination that the noise signal satisfies the threshold condition, that the patient is improperly wearing the wearable component; and
based on a determination that the patient is improperly wearing the wearable component, cause the sensing circuitry to suspend sensing the ECG signal; and
a fixation mechanism configured to electrically connect the computing device the one or more sensors.

17. The computing device of claim 16, wherein the one or more sensors comprises one or more strain gauges, wherein the sensing circuitry is configured to measure voltage values over time via the one or more strain gauges, and wherein the processing circuitry is further configured to:
determine, based on the measured voltage values, a respiration signal of the patient,
wherein the sensed signal comprises the respiration signal, and wherein the noise signal of the sensed signal comprises a noise signal of the respiration signal.

18. The computing device of claim 16, wherein the one or more sensors comprises one or more electrically active regions disposed along the wearable component, wherein the processing circuitry is configured to sense, via the sensing circuitry, the ECG signal from the one or more electrically active regions, wherein the sensed signal comprises the ECG signal, and wherein the noise signal of the sensed signal comprises a noise signal of the ECG signal.

19. The computing device of claim 18, wherein each of the one or more sense amplifiers is electrically connected to a corresponding electrically active region of the plurality of electrically active regions, and wherein each sense amplifier of the one or more sensed amplifiers is configured to sense the ECG signal from the skin of the patient.

20. The computing device of claim 16, further comprising a blower configured to connect to one or more expandable members disposed on the wearable component, and wherein the processing circuitry is further configured to:
based on the determination that the patient is improperly wearing the wearable component, engage the blower to expand the one or more expandable members to increase a contact force between the one or more sensors and the patient.

21. A method comprising:
sensing, by sensing circuitry of a computing module and via a plurality of electrically active regions disposed on a wearable component worn by a patient and configured to contact skin of the patient, an electrocardiogram (ECG) signal of a heart of the patient, wherein the wearable component is configured to encircle a portion of a torso of the patient, and wherein the wearable component comprises:
an electrically conductive fabric defining the plurality of electrically active regions along a longitudinal axis of the wearable component; and
a recess configured to retain the computing module;
determining, by processing circuitry of the computing module and based on the ECG signal, a noise signal within the ECG signal;
determining, by the processing circuitry, whether the noise signal satisfies a threshold condition;
determining, by the processing circuitry and based on a determination that the noise signal satisfies the threshold condition, that the patient is improperly wearing the wearable component; and
based on a determination that the patient is improperly wearing the wearable component, causing, by the processing circuitry, the sensing circuitry to suspend sensing the ECG signal.

22. The method of claim 21, wherein the sensing circuitry comprises a plurality of sense amplifiers, wherein each sense amplifier of the plurality of sense amplifiers is electrically connected to a corresponding electrically active region of the plurality of electrically active regions, and wherein causing the sensing circuitry to suspend sensing the ECG signal comprises:
transmitting a signal to the sensing circuitry to saturate the one or more sense amplifiers.

23. The method of claim 21, wherein the wearable component further comprises one or more strain gauges disposed along the longitudinal axis of the wearable component, and wherein the method further comprises:
sensing, by the sensing circuitry and via the one or more strain gauges, measured voltage values over time;
determining, by the processing circuitry and based on the measured voltage values, a respiration signal of the patient;
determining, by the processing circuitry, a noise signal of the respiration signal;
determining, by the processing circuitry, whether the noise signal of the respiration signal satisfies a threshold condition of the noise signal; and
based on a determination that the noises signal of the respiration signal satisfies a threshold condition of the noise signal, causing, by the processing circuitry, the sensing circuitry to suspend sensing the ECG signal.

24. The method of claim 21, wherein the wearable component comprises one or more expandable members in fluid communication with the recess and wherein when expanded the one or more expandable members are configured to increase a contact force between the skin and at least one of the plurality of electrically active regions, and wherein the method further comprises:
based on the determination that the noise signal satisfies the threshold condition, causing, by the processing circuitry, a blower disposed within the computing module to expand the one or more expandable members.
